(19) United States
(12) Patent Application Publication
(10) Pub. No.: US 2021/0275015 A1
(43) Pub. Date: Sep. 9, 2021

(54) ENHANCED OCULOMOTOR TESTING DEVICE AND METHOD USING AN ADD-ON STRUCTURE FOR A MOBILE DEVICE

(71) Applicants: Dovi Yellin, Kfar Saba (IL); Paul Morinville, Georgetown, TX (US)

(72) Inventors: Dovi Yellin, Kfar Saba (IL); Paul Morinville, Georgetown, TX (US)

(21) Appl. No.: 16/724,356

(22) Filed: Dec. 22, 2019

(51) Int. Cl. A61B 3/113; A61B 5/1171; A61B 5/00; A61B 5/16; G06K 9/00

(52) U.S. Cl. CPC A61B 3/113; A61B 5/1176; A61B 5/4863; G06K 9/00617; A61B 5/163; A61B 5/6898; A61B 5/0059

(57) ABSTRACT

According to an embodiment of the disclosure, an add-on structure communicates with the mobile device to provide supplemental features to the mobile device for the conducting of one or more oculomotor tests at high signal to noise ratio. The add-on structure includes a stimuli providing portion. An application loaded upon the mobile device coordinate the interaction of the mobile device and add-on structure in the conducting of the oculomotor test. At least one of the add-on structure or the mobile device includes an infrared (IR) sensor and an at least one IR illuminator.

### TECHNICAL FIELD

This disclosure is generally directed to the field of monitoring neurophysiological performance. More specifically, this disclosure is directed to an enhanced oculomotor testing device and method using an add-on structure to mobile device.

### BACKGROUND

Functions of the oculomotor nerve complex provide well-established methods for monitoring neurophysiologic performance. Known oculomotor tests for neurophysiological impairment detection include: the Near Point Convergence (NPC), the Horizontal Gaze Nystagmus (HGN), the Pupillary Light Reflex (PLR) checks and others. The validity of these tests for medical purposes has been substantiated in carefully devised scientific experiments, using highly accurate, complex and high-cost equipment. However, on the routine basis, when use of clinical gold-standard equipment is not attainable and commonplace state-of-art methods are in use, these tests are frequently not accurate enough, typically being administered manually by a human trained expert. In this scope, the human experts administering a test exert a subjective judgment, based on examinee performance, in order to provide a test score relative to some known norm, facilitating a diagnosis. Subsequently, taking advantage of recent technological development of mobile devices to overcome this subjective judgment, appears ideal. Yet, while wearable mobile devices are becoming more prelevant in the market-place, they have a variety of shortcoming in context of administering objective oculomotor tests, as detailed in the following section below.

### SUMMARY OF THE DISCLOSURE

Given the short-comings of using “bare” mobile devices (without necessary functional extension) in oculomotor testing as described herein, the disclosure provides a system and method for conducting enhanced oculomotor testing using an add-on structure to mobile device.

According to an embodiment of the disclosure, an add-on structure communicates with the mobile device to provide supplemental features to the mobile device for the conducting of one or more oculomotor tests. The add-on structure includes a stimuli providing portion as well as additional sensors for collecting higher quality signal even under demanding environmental light conditions. An application loaded upon the mobile device coordinates the interaction of the mobile device and add-on structure in the conducting of the oculomotor test. At least one of the add-on structure or the mobile device includes an infrared (IR) sensor and at least one IR illuminator (both sensor and illuminator at preferred operational range centered around wavelength of approximately 850 nm). Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. The phrase “at least one of,” when used with a list of items, means that different combinations of one or more of the listed items may be used, and only one item in the list may be needed. For example, “at least one of: A, B, and C” includes any of the following combinations: A; B; C; A and B; A and C; B and C; and A and B and C. Definitions for certain words and phrases are provided throughout this patent document, those of ordinary skill in the art should understand that in many if not most instances, such definitions apply to prior, as well as future uses of such defined words and phrases.

### BRIEF DESCRIPTION OF THE DRAWINGS

For a more complete understanding of this disclosure and its features, reference is now made to the following description, taken in conjunction with the accompanying drawings, in which:

FIGS. 1A and 1B show an integrated device, according to an embodiment of the disclosure;

FIG. 2 shows the location of the stimuli and how the screen can be moved toward a subject for a nearing or retracting dot scenario test (as in a NPC test);

FIG. 3 shows an example use of the device of FIGS. 1A, 1B, and 2 with a subject, according to an embodiment of the structure;

FIGS. 4A, 4B, and 4C illustrates varying holders and corresponding features that may be utilized, according to embodiments of the disclosure;

FIG. 5 illustrates another aspect of capturing distance of a subject with reference to the mobile device—according to embodiments of a disclosure;

FIGS. 6A and 6B additional measurement techniques, according to embodiments of the disclosure;

FIG. 7 show a simplified block diagram illustrative of a communication system that can be utilized to facilitate communication between endpoint(s) through a communication network, according to particular embodiments of the disclosure;

FIG. 8 is an embodiment of a general-purpose computer that may be used in connection with other embodiments of the disclosure to carry out any of the above-referenced functions and/or serve as a mobile computing device for endpoint(s); and

FIGS. 9A and 9B shows an additional configuration of an integrated device 950, providing an implementation example of FIG. 1A add-on structure 100, according to an embodiment of the disclosure.

### DETAILED DESCRIPTION

The FIGURES described herein, and the various embodiments used to describe the principles of the present disclosure in this patent document are by way of illustration only and should not be construed in any way to limit the scope of the disclosure. Those skilled in the art will understand that the principles of the present disclosure may be implemented in any type of suitably arranged device or system. Additionally, the drawings are not necessarily drawn to scale.

As indicated earlier, functions of the oculomotor nerve complex provide well-established methods for monitoring neurophysiologic performance. Known oculomotor tests for neurophysiological impairment detection include: the Near Point Convergence (NPC), the Horizontal Gaze Nystagmus (HGN), the Pupillary Light Reflex (PLR) checks and others. The validity of these tests for medical purposes has been substantiated in carefully devised scientific experiments, using highly accurate, complex and high-cost equipment. However, on a routine basis, when use of clinical gold-standard equipment is not attainable and commonplace state-of-art methods are in use, these tests are frequently not accurate enough, typically being administered manually by a human trained expert. In this scope, human experts exert a subjective judgment, based on examinee performance, in order to provide a test score relative to some known norm, facilitating a diagnosis.

Mobile devices are becoming more and more powerful processing tools; however, by themselves, they are still insufficient for reliable oculomotor tests. As a non-limiting example, even mobile devices with a high definition camera (FHD—1920×1080 pixels or even UHD—3840×2160 pixels resolution) have insufficient ability to capture eye-movement in low light conditions. When such mobile devices attempt to add light (e.g., from a flash) to rectify the low-light conditions, the prolonged light distort the testing. Light reflection and glints add an additional source of noise to images obtained by normal light cameras. In the alternative, while certain mobile devices may have infrared features, such infrared features are not designed in the image context of eye-marker extraction and are thus unsuitable for oculomotor tests.

Given these difficulties, embodiment of the disclosure provide testing using a standard mobile device, such as a smartphone, supplemented as necessary with an add-on structure that has a two-way communication with the standard mobile device. When combined with the application installed on the mobile device, the combined device enables accurate and consistent administration of oculomotor functional tests, known to provide a well-established method for monitoring neurophysiological performance. In particular, the combined device in specific embodiments enables the detection of neurophysiological impairment measured by visual pursuit tracking, pupillometry, vergence, eye-lid tremor, blink metrics (such as the mean and variance of blink rate, duration, closure speed etc.) and gaze nystagmus tests. In certain embodiments, the combined device may be used to automate and facilitate these oculomotor tests to detect impairment due to consumption of toxic substances (such as drugs and alcohol), as well as the monitoring of impairment due to mild traumatic brain injuries (as in sports concussions) and sleep deprivation. While certain impairments will be described herein, it should be understood that such are intended as being non-limiting examples. The same disclosure herein may also be used for other types of impairments—including those associated with after-arising technologies and techniques and non-neurological related impairments (e.g., other medical or biological related impairments). Also, while certain tests are referenced, other tests may be performed.

The following literature provides example of oculomotor tests and feedback for determining whether a condition exists:
1. Kulkarni et al (2015). Ocular manifestations of head injury: a clinical study. Eye 19(12):1257-63
2. Miller (2006) Neuro-ophthalmologic manifestations of psychogenic disease. Semin Neurol. 26(3):310-20
3. Patel and Lundy (2002) Ocular manifestations of autoimmune disease. Am Fam Physician. 15; 66(6):991-8
4. Cohen et al (1989) Convergence Insufficiency in Brain Injured Patients. Brain Inj: 3: 187-191.
5. Bruke et al (1993) Convergence Insufficiency in Thyroid Eye Disease. J Pediatr Ophthalmol Strabismus. 30: 127-129.
6. Biousse et al. (2004) Ophthalmologic features of Parkinson's Disease. Neurology 62: 177-180.
7. Ciuffreda et al (2017). Understanding the effects of mild traumatic brain injury on the pupillary light reflex. Concussion. 2(3).
8. Garner et al (2018). Blink reflex parameters in baseline, active, and head-impact Division I athletes. Biomedical Engineering.
9. Serra and Leigh (2002) Diagnostic value of nystagmus: spontaneous and induced ocular oscillations. Journal of Neurology, Neuros. & Psych. 73:615-618.
10. Poynter (2017). Pupil-size asymmetry is a physiologic trait related to gender, attentional function, and personality. Laterality. 22(6):654-670.
   Again, while such example tests are described, other tests—including those that have yet to be developed—may avail from the disclosures herein.

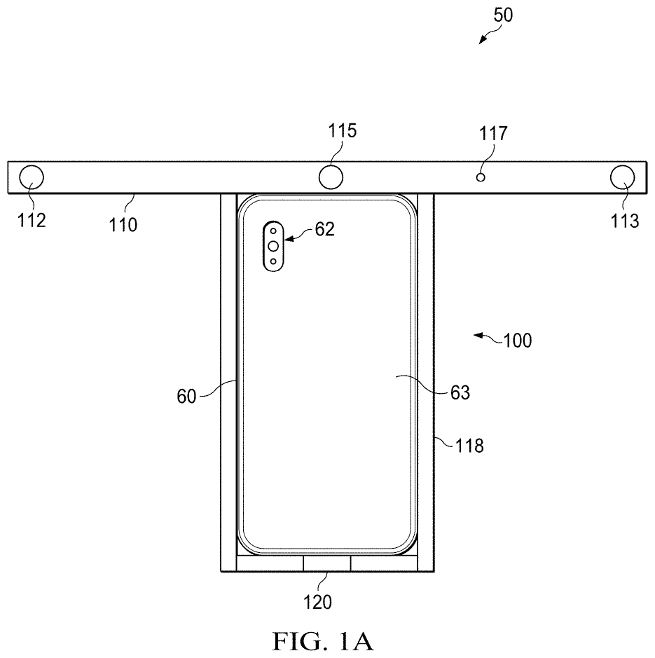

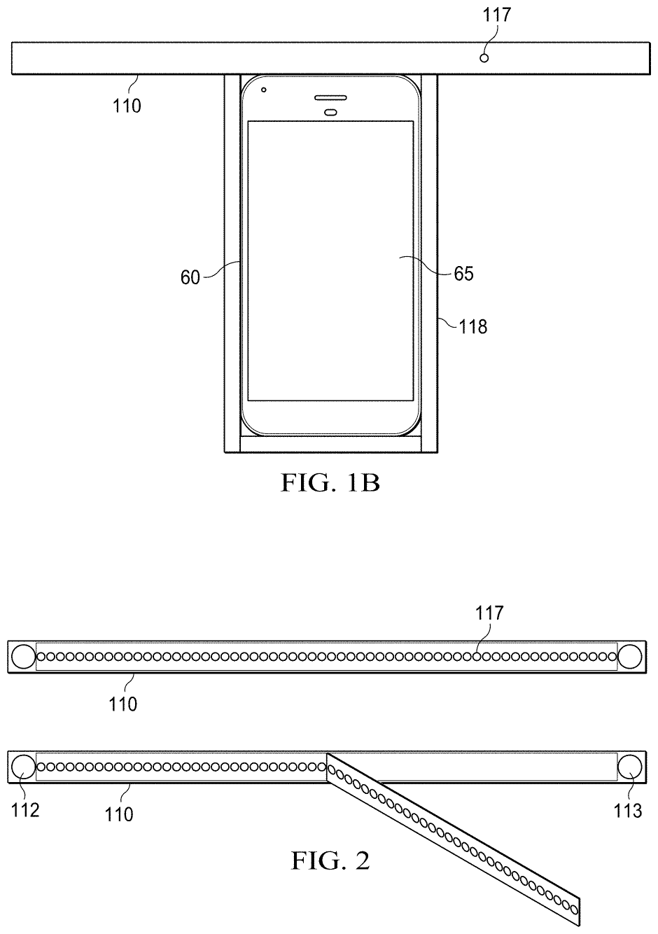

FIGS. 1A and 1B show an integrated device 50, according to an embodiment of the disclosure. FIG. 1A is a back side of the device 50 that would face a subject being tested whereas FIG. 1B is the front-side of the device that faces an administrator who is running tests. The integrated device 50 includes a mobile device 60 and an add-on structure 100.

In particular configurations, the mobile device 60 may be a commercially available smartphone—including those currently available from manufactures such as Apple (e.g., the iPhone), Google (e.g., Pixel phones), Samsung (e.g., Galaxy Phones), and others. In other configurations, the mobile device 60 may include other commercially devices such as iPads or tablets. Also, in particular configurations, the mobile device 60 may include smart watches. A general description of capabilities of the mobile device 60 is described with reference to FIG. 8, including features such as processors, cameras, operating systems, RAM, communication capability, and the like.

In this particular configuration, the mobile device 60 is a smartphone that includes one or more cameras 62. The particular one or more cameras 62 in this configuration is a backside camera. The backside 63 of the mobile device is also shown.

The add-on structure 100 is generally designed to provide additional functionality to that may not exist on the mobile device 60. While described in particular configurations as providing “additional” features, in particular configurations the add-on structure 100 may provide features either that are technically redundant of feature of the mobile device 60. This redundancy may either satisfy a universal design to include features that are absent in most mobile devices used or to provide the quality expected for proper operation of the device 50. Further examples will be provided below.

As shown in the configuration of FIG. 1, the add-on structure 100 includes a screen 110, an IR sensor 120, and a clamping portion 118.

Left and right infrared (IR) illuminators 112, 113 are coupled to the screen 110 to illuminate a subject's eye and reflect as appropriate. These IR illuminators may be placed in a different locations on the add-on a 110, or coupled to the screen 110, are stimuli-providing portions 117. While only one is shown, multiple stimulation portions may exist as discrete portions along the screen. The stimuli portions 117 can create a moving dot representation on the screen, for example, to which an eye focuses as discussed in further may be utilized. The stimuli portions 117 may be created in any suitable manner—including discrete light emitting diodes.

Also, as either part of the screen 110 or coupled to the screen 110 is a light 115, which will be discussed in further details below.

The IR sensor 120 captures reflection from the illumination by the left and right infrared IR illuminators 112, 113. In certain configurations, the IR sensor 120 is high-definition. In particular, it has a resolution of at least 1920 by 1080 pixels. In other configurations, the resolution may be even higher. Optimal sensitivity of the sensor, in a typical embodiment, is at wavelength of 850 nm.

The clamping portion 118 generally provides an ability to hold portions of the mobile device 60—allowing an interconnection between the add-on structure 110 and the mobile device. In some configurations, any suitable connection mechanisms may be utilized to facilitate the coupling of the phone. And, in certain configurations, the clamping portions facilitate the handling of the integrated device 50—including any suitable features that assist such handling. As one non-limiting example, the clamping portions may be coated with material that allows a better grip.

While one particular conjuration is shown in FIG. 1, other descriptions below provide alternative coupling mechanisms for connecting the mobile device to the add-on structure.

While certain components have been described herein, in other configuration the add-on structure 100 may include more or less components. In certain configurations, the components on the add-on structure 100 may depend on the functionality and/or components on the mobile device 60 whereas in other configurations, the components add-on structure 100 may not depend on features of the mobile device 60. In the latter configurations, the add-on structure 100 may have features that are redundant to certain features on the mobile device 60. As a non-limiting example, a light 115 may be redundant of a flashlight also on the phone; however, the reason for this perceived redundancy is explained below.

As either part of the clamping portion 118 or the add-on structure 110 more generally, any of a variety of two-way communication technologies may be utilized to allow two-way communication between the mobile device 60 and the add-on structure 100. Non-limiting example technologies include Bluetooth, Wifi, Wired (e.g., USB-C), and others. Yet other suitable components for each will become apparent after review of the disclosure.

Although not explicitly shown here, certain configurations of the add-on structure 110 may additionally include a power source (e.g., batteries or other energy storage). In other configurations, the add-on structure 110 may principally receive power from the mobile device 60. As a non-limiting example, the add-on structure 110 may receiver power through a USB-C connection it has with the mobile device 60. Yet other manners of powering the add-on structure may alternatively be utilized.

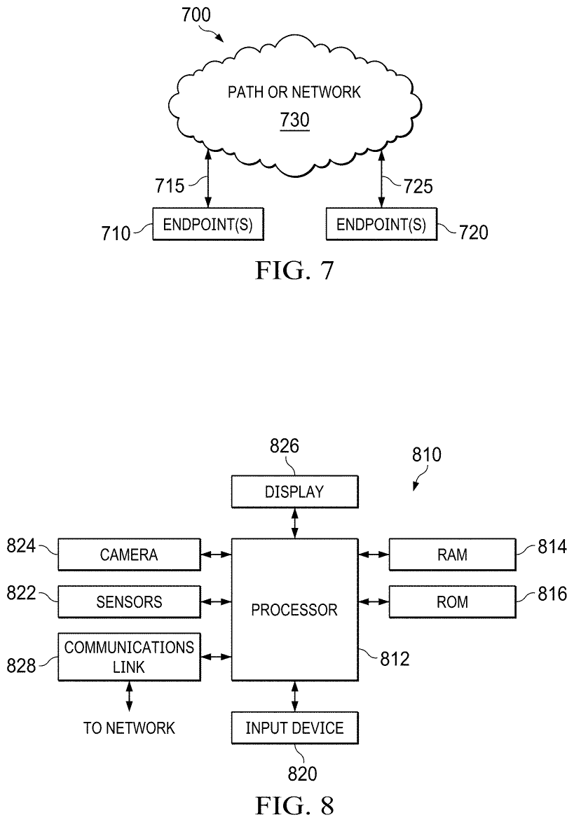

In general operation, a two-way communication exists between the add-on structure 110 and the mobile device 60—passing instruction and information back and forth to conduct the oculomotor tests. As an example, instructions on timing and pace of stimulation provided by mobile device 60 to add-on structure 110 on the one hand, and IR sensor information from the later back to the former. While processing may be performed locally on the mobile device 60, in other configurations, the mobile device 60 may off-load some, none, or all of the processing to a remote device (e.g., servers) using communication capabilities of the mobile device 60. Non-limiting example of communication are described with reference to FIG. 7.

While certain sensors will be described with reference to certain embodiments, other sensors may alternatively be utilized. For example as described below, Time-of-Flight (ToF) cameras may be utilized. LIDAR systems (including associated sensor) may also be utilized. Yet other sensor may alternatively be utilized while remaining in the scope of this disclosure, including but not limited to, those associate with photogrammetry.

With reference to FIG. 1B, the front side 65 of the mobile device 60 may be used for an administrator of the test. In particular configurations, an application or “app” may be loaded on to the mobile device 60 and allow the administrator to interact with the touchscreen features typical of mobile devices 60 in the administration of different tests. Such apps may be loaded through app stores such as Google Play, the Apple App store or others. In particular configurations instructions may be provided to the administrator on what needs to be done next. Or, if appropriate readings were not obtained, instructions to repeat a test. The stimuli 117 may have at least a faded portion showing through to allow an administrator to see the location of the stimuli. However, the stimuli 117 need not be the same on both sides—serving only as an indicator on side of FIG. 1B.

The app installed on the phone may correspond to an account where information can be stored and recalled (e.g., on a different mobile device 60) by simply logging (or authenticating as appropriate) into an account. Any suitable authentication protocol may be used for such embodiments.

In particular configurations, portions or all of tests being conducted may be automated. In particular, an administrator upon selecting in the app a particular test to be performed may initiate the testing with the mobile device in an automated preconfigured fashion. In particular configurations, an automatic mode may be preferred for accuracy and consistency. For example, an administrator may simply hit a “start test” button and let the system do its job without subjective interpretations by an administrator. Such a configuration may be particularly helpful with newer users that need little or no training to start administering a test. Also, in particular configurations, little or no human intervention may be required for a test at all. In automated testing, using dynamic feedback received from measurement of the eye, the moving dots can start moving after participant eye calibration phase is done. When a certain movement of the eye needs to be reviewed more carefully, the application can change the speed and movement of the moving dot representation.

In the automated operations, the processing for dynamic feedback to change the test may be performed locally, remotely, or combination of both.

In other configurations, an administrator may be allowed to move the dots as desired using either a virtual touch-screen slider on the front side 65 or by touching the back-side of the screen. In such configurations, seasoned Drug Recognition Evaluators (DRE) may have a desire to have more control over the testing processes.

According to one non-limiting example, the add-on device may have the below specifications. While such example specifications are provided, it should be expressly understood that the disclosure is not limited to such examples. Different specifications can be used—including those with larger or smaller values than those provided. The disclosure should not be interpreted as being limited to such examples.

The length of the screen 110 may be approximately 60 cm for a horizontal reach of 30 cm of tracking distance per each side (left and right). Assuming the device 50 is held at 30 cm distance from examinee, a 45-degree angle would form for horizontal gaze nystagmus (HGN) testing.

The width of the screen 110 may be approximately 1 cm. Such a width can facilitate a good view of a dot (constructed by led) moving horizontally from edge to edge at preconfigured pace. The dot diameter can be approximately 0.5 cm and green in color. Other colors can alternatively be used.

In an automatic mode, a complete cycle for a test (e.g., one full movement of the dot from center all the way to left, back to center, then all the way to right and finally back to center) may be completed in approximately 5 seconds. However, the timing of a cycle can be configurable. Also, as referenced above, in particular configurations, the testing can be manual.

The thickness of the add-on structure 100 can be approximately 0.5 cm—providing robustness and preventing the add-on structure 100 from bending or breaking.

The light 115 may be designed for optimized pupillary light reflex (PLR)-related light flash, so that the pupil responds clearly to the stimuli, but without causing any long-term damaging effect to the participant's eyes.

In particular configurations, portions of the device may be foldable allowing for convenient carrying. For purpose of the near point convergence (NPG) test, a segment of the screen 110 have the ability to tilt perpendicularly (facing the examinee), facilitating a nearing or retracting dot scenario (as shown in FIG. 2).

FIG. 2 shows the location of the stimuli 117 and how the screen 110 can be moved toward a subject for a nearing or retracting dot scenario test. In a similar fashion to movement of the screen 110, in particular configuration, the screen 110 may be folded for storage. Such folding may occur in any suitable manner.

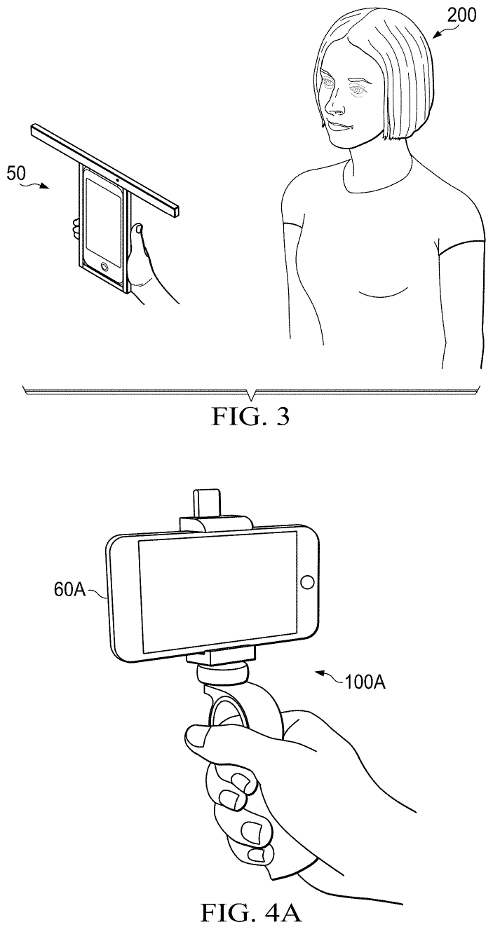

FIG. 3 shows an example use of the device of FIGS. 1A, 1B, and 2 with a subject 200, according to an embodiment of the structure. The light 115 may be a bright light-emitting diode. Such a light 115 can facilitate PLR testing. In particular configurations, a 450 nm wavelength light may be used for approximately one second. In other configurations, different wavelength and different timing may be used.

The use of IR sensor 120 and IR illuminators 112, 113 would enable overcoming smartphone normal light camera limitations relating to: dynamic lighting conditions (brightness, backlight, reflections), dark eyes (lack of contrast for some users), dynamic background, and the like.

Providing such features allows the device 50 to benefit from capturing the image with an IR camera of high resolution in order to apply both full face and eyes image processing techniques, as well as known IR-oriented pupil extraction methods.

Operationally, the information captured by the IR sensor 120, alone, may be utilized for detecting eye movement. In such configurations, the one or more cameras 62 of the mobile device 60 may still be used for alignment and testing. In other configurations, the eye movement may be detected by a combination of the IR sensor 120 and the one more cameras 62 of the mobile device 60. While one IR sensor 120 is shown in this configuration, more than one can be used in other configurations. Also, as described herein, different types of sensors and/or cameras may be present on either the add-on structure 100 or mobile device 60.

The following are non-limiting example oculomotor tests that may be conducted by the integrated device 50, according to embodiments of the disclosure. While such test are examples, the integrated devices herein (including integrated device 50) may be utilized for other tests—including after-arising tests that are later developed.

Horizontal Gaze Nystagmus (HGN) is a biphasic ocular oscillation alternating a slow eye movement, or smooth pursuit, in one direction and a fast jerky eye movement, or saccadic movement, in the other direction. The velocity of the slow phase eye movement (SPEV) and the fast phase eyes velocity (FPEV) are related to each other and can be considered as a measurement of the efficiency of the system stimulus/response. Particular embodiments may also use vertical gaze nystagmus (VGN) testing.

Near Point Convergence (NPC) provides a measure of pursuit convergence by testing closer and closer points of gaze on an object (such as a finger or pencil) held in front of the eyes, until one of the eyes is no longer able to fixate on the object. As a result, this point is the near point of the convergence. Normal near point of convergence is about 6-10 cm for normal eyes but the convergence recovery point (CRP) typically extends to 15 cm. If the NPC is more than 10 cm there's sign of poor convergence. Results are noted in terms of NPC, CPR; for example, NPC 7 cm, CRP 12 cm.

Pupillary Light Reflex (PLR) is defined by systematic constriction of both pupils in response to the onset of a time-limited light stimulus, followed by a refractory dilation period after stimuli offset. The pupil size must change by a non-trivial amount within a specific time frame and should change in both eyes. PLR is a well-established measurement in the management and prognosis of patients with acute brain injuries, in conjunction with other clinical parameters such as age, mode of injury and Glasgow Coma Scale [79-81]. Typical light stimuli parameters are white (multichromatic) light or blue light (at 465 nm wavelength) with a duration of 1 s and typical luminance of 0.001 candelas/square meter (cd/m2). [Ref: Kelbsch et al. (2019) Standards in Pupillography. Frontiers in Neurology. 10: 129.]

In particular applications, for example, those used in sports, the camera feature of the phone may be used to create baseline profile for particular users. The identity of the user may be manually selected, facially recognized (and matched—e.g., using the camera), biometrically identified with a fingerprint, or matched through iris recognition. Oculomotor tests may be performed to create and store (either locally or remotely, for example in the cloud) a baseline for a particular subject. Then, for example, when a brain injury (e.g., a concussion is suspected), the same subject can be tested again—recalling the baseline stored event—and comparing the new neurophysiological results with baseline results. Because of its interconnectivity with remote computers, a user may simply use any suitable mobile device (e.g., mobile device 60), log-in to an account to recall data, and strap-on the add-on instrument to quickly perform test on the side-line of an event.

In another application, for example, oculomotor tests used in detecting drug impairment, the extra features allow testing in diversified environments (e.g., night-time). A police officer may simply be provided the add-on structure 100 with the corresponding application for fitting on an existing mobile device such as a phone or tablet (e.g., iPad). In addition to the oculomotor testing itself, a recordation (e.g., evidence) of the testing can be captured using the video features of the mobile device 60. Both the test results and video evidence can be stored locally or uploaded to the cloud (e.g., remove servers) for later use.

In particular configurations, as referenced herein, such as those used by Drug Recognition Evaluators (DRE), a DRE may desire to manually control the stimuli. Thus, rather than certain tests being automatic, the DRE can manually control the speed and location of the stimuli. Where the screen (device 110) is used, this can be accomplished using, for example, either a slider on the administrative screen for NPC or HGN tests.

In particular configurations, an aggregation of data from multiple testing of a subject or multiple subjects can be cross-referenced to determine patterns associated with different conditions—either to enhance detection of condition or for further research.

In the different type of use cases, the application can provide immediate feed-back as necessary to the administrator as a condition (e.g., concussion likely, drug detected, or the like). The feedback may consult cloud-computing (potentially applying predictions of machine-learning models) as necessary if additional processing power is necessary beyond that provided by the mobile device. One of ordinary skill in the art will recognize the benefit-cost analysis between local and cloud computing. As a non-limiting example, cloud computing provides more processing power, but may take longer to access. Regardless of whether cloud computing is used for instantaneous feedback, following a test, the results can later be sent to the remote computers (e.g., the cloud) for storage and/or later analysis. As an example of storage, later testing may reveal that the very particular immediate conditions for a user that yielded a concussion. Such information may be used for training machine learning models for later use on a user-specific basis as an indicator of a concussion.

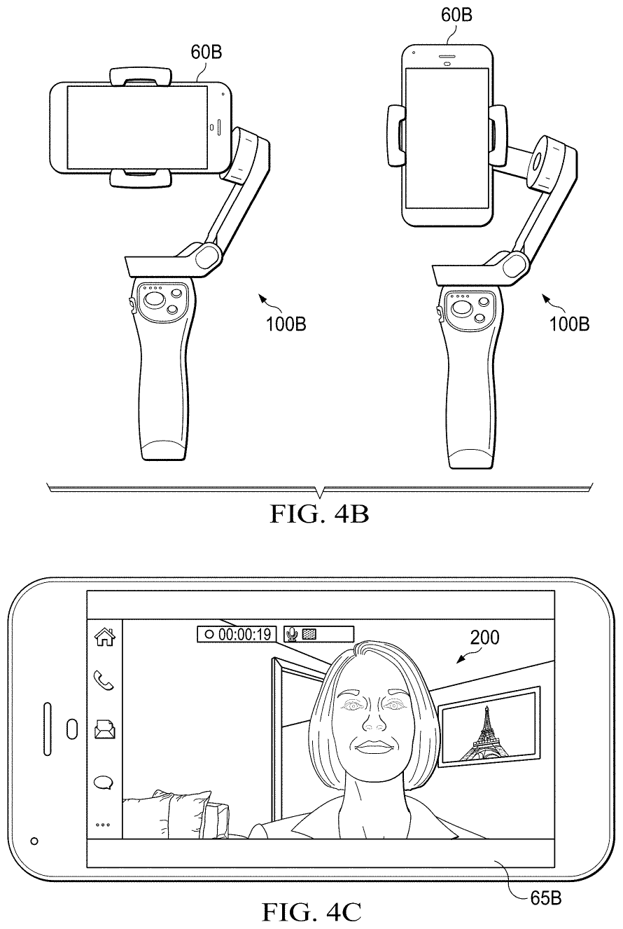

FIGS. 4A, 4B, and 4C illustrates varying holders and corresponding features that may be utilized, according to embodiments of the disclosure. While such holders are shown, it should be understood that other holders may additionally be utilized and avail from embodiment of the disclosure.

With reference to 4A, the mobile device 60A is shown as being tilted. As part of the add-on structure 100A, a holder or handle is provided to an administrator to facilitate stable posture and convenient administration of tests.

With reference to FIG. 4B, the mobile device 60B is mounted on an add-on structure 100B that includes a gimbled-device—to keep the mobile device steady, even through small movements. The gimballed-device may support both vertical and horizontal positioning of the mobile device. In particular configurations, the gimbals may be mechanically operated to keep such stability. And, according to certain configurations, the test subject can be selected as an active tracking object—with communication feedback to mechanical gimbals to make sure object is kept in focus. Such a selection of features is shown with reference to FIG. 4C on the front side screen 65. In particular, a face of the subject 200 is selected (as indicated by the bracket surround the face) and mechanic gimbals keep the bracketed face in focus—even through slight movements of the mobile device. An example mechanical gimbled-device that receives a mobile device and has active tracking features is sold by DJI of Shenzhen, China under the name OSMO MOBILE. Yet other gimbaled devices or mechanical stabilizers may alternatively be utilized.

Feedback for the gimbaled device can be obtained from the one or more cameras of the mobile device, the IR sensor, another sensor, or a combination of the proceeding.

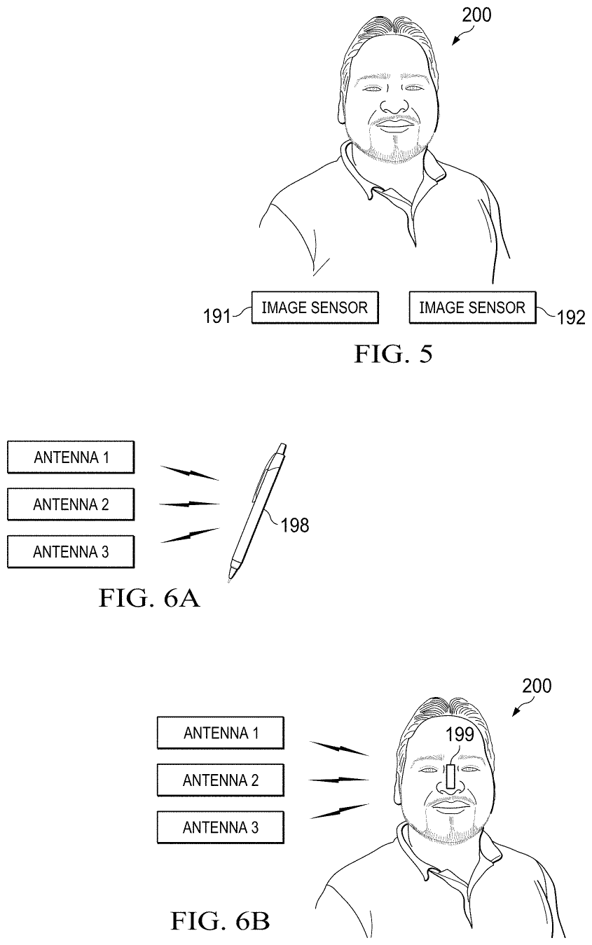

FIG. 5 illustrates another aspect of capturing distance of a subject with reference to the mobile device—according to embodiments of a disclosure. FIG. 5 illustrates how one may triangulate an image distance to a subject 200 using at least two image sensors 191, 192. In particular configurations, the integral camera from the mobile device and the IR sensor (residing at a known distance from one another) may be used to determine the distance of the subject. In newer mobile devices that have multiple cameras, the integral functions of such mobile device which use their sensors for distances may be utilized. As non-limiting examples, newer applications on such mobile devices have measuring features to measure, for example, the distance between objects—using built-in sensor. Such features of the phone (where they exist on the mobile device) can also be used.

In alternative configurations, Time-of-Flight (ToF) cameras may be utilized—either obtained from mobile device built-in-features or using one from the add-on structure. ToF camera sensor manufacturers/devices include, but are not limited to AMS/Heptagon, TeraRanger One, ASC TigerCub, Riegl, and Lucid/Helios.

Like the sensing of the distance to the test subject 200, certain configurations may use the same sensor (combination of sensors in the mobile device and newly added ones with the add-on structure) to similarly measure the distance of a stimuli pen (or stimulate light pen) from the device for an understanding of the relative angle of the eye being measure. In such configurations, a screen as described in FIGS. 1A, 1B, 2, and 3 may not be utilized.

A yet another sensor that may be utilized in certain configurations are one or more 360-degree cameras that use fish effects to capture a large area. Such cameras may serve a dual purpose in some configurations of capturing both a stimuli pen and the subject—for distance determination. Example small 360-degree camera are sold by Insta360 of Shenzhen, China under the name INSTA360.

FIGS. 6A and 6B illustrate additional measurement techniques, according to embodiments of the disclosure. FIGS. 6A and 6B show the use of propagated electromagnetic waves to, for example, triangulate and detect both the distance of and relative angle between an object (e.g., stimuli pen 198) and the test subject (e.g., using an emitter 199 that may be placed on a nose of the test subject 200).

While one antenna can determine distance, two or three (or even four) can better approximate. Three or shown in FIGS. 6A and 6B. The antennas themselves can either be completely in the add-on structure or at least some can be on the antennas of the mobile device. One of ordinary skill in the art will recognize how the same signal propagated and received by three separate antennas can be used to determine distance and direction. Such techniques are commonly used in a much larger scale for GPS and Cell-tower triangulation. Here, the same techniques are used for much smaller scale triangulation to achieve millimeter level accuracy.

With a configuration such as that shown, all three items (device, subject, and stimuli) are known over time in a three-dimensional space relative to one another and can be correlated with the eye movement.

In particular configurations, different technologies described herein may be combined.

FIG. 7 is a simplified block diagram illustrative of a communication system 700 that can be utilized to facilitate communication described herein. Each item associated with a communication is generically described as endpoint 710, 720 which communicate through a path or communication network 730. With reference to the FIGURES described above, one endpoint may be the mobile device whereas the other endpoint may be add-on structure. Likewise, one endpoint may be the mobile device whereas the other endpoint may be a cloud-computing processor, storage, or both.

When referencing communication, for example, showing arrows or “clouds,” or “networks,” any of such communication may occur in the manner described below or other manners. Or, a communication can be local, for example, over USB-C or Bluetooth in which. Likewise, the endpoints may generally correspond to any two particular components described (or combination of component) with another component or combination of components.

As used herein, “endpoint” may generally refer to any object, device, software, or any combination of the preceding that is generally operable to communicate with and/or send information to another endpoint. In certain configurations, the endpoint(s) may represent a user, which in turn may refer to a user profile representing a person. The user profile may comprise, for example, a string of characters, a user name, a passcode, other user information, or any combination of the preceding. Additionally, the endpoint(s) may represent a device that comprises any hardware, software, firmware, or combination thereof operable to communicate through the communication path or network 730.

Examples of an endpoint(s) include, but are not necessarily limited to those devices described herein, a computer or computers (including servers, applications servers, enterprise servers, desktop computers, laptops, netbooks, tablet computers (e.g., IPAD), a switch, mobile phones (e.g., including IPHONE and Android-based phones), networked televisions, networked watches, networked glasses, networked disc players, components in a cloud-computing network, or any other device or component of such device suitable for communicating information to and from the communication path or network 730. Endpoints may support Internet Protocol (IP) or other suitable communication protocols. In particular configurations, endpoints may additionally include a medium access control (MAC) and a physical layer (PHY) interface that conforms to IEEE 801.11. If the endpoint is a device, the device may have a device identifier such as the MAC address and may have a device profile that describes the device. In certain configurations, where the endpoint represents a device, such device may have a variety of applications or “apps” that can selectively communicate with certain other endpoints upon being activated.

The communication path or network 730 and links 715, 725 to the communication path or network 730 may include, but is not limited to, a public or private data network; a local area network (LAN); a metropolitan area network (MAN); a wide area network (WAN); a wireline or wireless network (WIFI, GSM, CDMA, LTE, WIMAX, BLUETOOTH or the like); a local, regional, or global communication network; portions of a cloud-computing network; a communication bus for components in a system; an optical network; a satellite network; an enterprise intranet; other suitable communication links; or any combination of the preceding. Yet additional methods of communications will become apparent to one of ordinary skill in the art after having read this specification. In particular configuration, information communicated between one endpoint and another may be communicated through a heterogeneous path using different types of communications. Additionally, certain information may travel from one endpoint to one or more intermediate endpoint before being relayed to a final endpoint. During such routing, select portions of the information may not be further routed. Additionally, an intermediate endpoint may add additional information.

Although endpoint generally appears as being in a single location, the endpoint(s) may be geographically dispersed, for example, in cloud computing scenarios. In such cloud computing scenarios, and endpoint may shift hardware during back up. As used in this document, “each” may refer to each member of a set or each member of a subset of a set.

When the endpoints(s) 710, 730 communicate with one another, any of a variety of security schemes scheme may be utilized. As an example, in particular embodiments, endpoint(s) 720 may represent a client and endpoint(s) 730 may represent a server in client-server architecture. The server and/or servers may host a website. And, the website may have a registration process whereby the user establishes a username and password to authenticate or log in to the website. The website may additionally utilize a web application for any particular application or feature that may need to be served up to web site for use by the user.

A variety of embodiments disclosed herein may avail from the above-referenced communication system or other communication systems.

FIG. 8 is an embodiment of a general-purpose computer 810 that may be used in connection with other embodiments of the disclosure to carry out any of the above-referenced functions and/or serve as a computing device for endpoint(s) 710 and endpoint(s) 720.

General purpose computer 810 may generally be adapted to execute any of the known OS2, UNIX, Mac-OS, Linux, Android and/or Windows Operating Systems or other operating systems. The general-purpose computer 810 in this embodiment includes a processor 812, random access memory (RAM) 814, a read only memory (ROM) 816, input device 818, one more camera(s) 824, input devices 820, sensors 822, a display 826 and a communications link 928. In other embodiments, the general-purpose computer 810 may include more, less, or other component parts. Embodiments of the present disclosure may include programs that may be stored in the RAM 814, the ROM 816 or other storage devices and may be executed by the processor 812 in order to carry out functions described herein. The communications link 828 may be connected to a computer network or a variety of other communicative platforms including, but not limited to, a public or private data network; a local area network (LAN); a metropolitan area network (MAN); a wide area network (WAN); a wireline or wireless network; a local, regional, or global communication network; an optical network; a satellite network; an enterprise intranet; other suitable communication links; or any combination of the preceding. In certain configurations, antennas may be utilized.

The camera may include a variety of types of camera—capturing either images or infrared reflections. The sensor may include any suitable sensor for capturing environmental parameters or other items for processing and feedback.

The input device 820 may include a tactical input device associated with the display 826, a keyboard, a mouse, or other input device.

Although FIG. 8 provides one embodiment of a computer that may be utilized with other embodiments of the disclosure, such other embodiments may additionally utilize computers other than general purpose computers as well as general purpose computers without conventional operating systems. Additionally, embodiments of the disclosure may also employ multiple general-purpose computers 810 or other computers networked together in a computer network. The computers 810 may be servers or other types of computing devices. Most commonly, multiple general-purpose computers 810 or other computers may be networked through the Internet and/or in a client server network. Embodiments of the disclosure may also be used with a combination of separate computer networks each linked together by a private or a public network.

Several embodiments of the disclosure may include logic contained within a medium. In the embodiment of FIG. 87, the logic includes computer software executable on the general-purpose computer 810. The medium may include the RAM 814, the ROM 816, or other storage structure. In other embodiments, the logic may be contained within hardware configuration or a combination of software and hardware configurations.

The logic may also be embedded within any other suitable medium without departing from the scope of the disclosure.

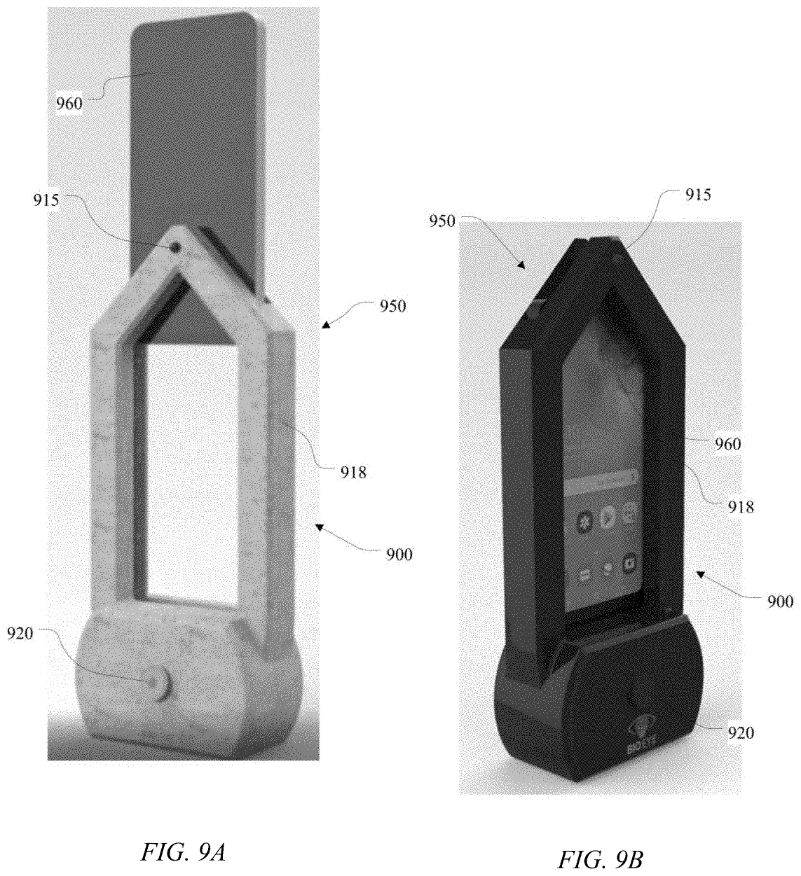

FIGS. 9A and 9B shows an additional configuration of an integrated device 950, according to an embodiment of the disclosure. The integrated device 950 may include features similar to those described above with reference to FIGS. 1A and 1B—including an add-on structure 900, a mobile device 960, a clamping portion 918, an IR sensor 920, and a light 915. The integrated device 900 may operate in a similar manner to the integrated device 50 above—including use of an application loaded on the mobile device.

Additionally, operationally, the integrated devices 950 may operate in accordance with any of the configurations described herein. With specific FIGS. 5, 6A, and 6B, the referenced sensor and/or antennas may be incorporated in any portion of the add-on structure 900. Similarly, a stimuli pend 198 may be used. Or, a screen 110 may be used. Yet, alternatively, other stimuli objects in communication with either the mobile device 960 or add-on structure 900 may be used. Suitable communications include, but are not limited to, Bluetooth and Wi-Fi. Others are described with refence herein that may also be used.

It will be understood that well known processes have not been described in detail and have been omitted for brevity. Although specific steps, structures and materials may have been described, the present disclosure may not be limited to these specifics, and others may substitute as is well understood by those skilled in the art, and various steps may not necessarily be performed in the sequences shown.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. An integrated device for conducting oculomotor tests, the integrated device comprising:
a mobile device,
an add-on structure that communicates with the mobile device to provide supplemental features to the mobile device for the conducting of one or more oculomotor tests, the add-on structure including a stimuli providing portion; and
an application loaded upon the mobile device to coordinate the interaction of the mobile device and add-on structure in the conducting of the oculomotor test.

2. The integrated device of claim 1, wherein
wherein at least one of the add-on structure or the mobile device includes an infrared (IR) sensor and an at least one IR illuminator that provides information directly or indirectly to the application.

3. The integrated device of claim 2, wherein
the IR sensor includes a resolution of at least 1920 by 1080 pixels,
the IR sensor is part of the add-on structure, both IR sensor and illuminator with operational range centered at wavelength of 850 nm

4. The integrated device of claim 1, wherein
the application is configured to communicate with remote computers for an account where information can be stored and recalled.

5. The integrated device of claim 1, wherein
the application instructs a user and automates at least a portion of the one or more oculomotor test through feedback received through one or more sensors on the integrated device.

6. The integrated device of claim 1, wherein the stimuli-providing portion includes a plurality of light emitting diodes positioned on a screen, facilitating a Horizontal Gaze Nystagmus (HGN) procedure.

7. The integrated device of claim 5, wherein the screen is foldable, facilitating a Near Point Convergence (NPC) procedure.

8. The integrated device of claim 5, wherein the stimuli-providing portion includes an optimized pupillary light reflex (PLR)-related light flash.

9. The integrated device of claim 1, wherein the integrated device is configured to recognize an individual.

10. The integrated device of claim 8, wherein the integrated device recognizes an individual using facial recognition.

11. The integrated device of claim 8, wherein the integrated device recognizes using iris recognition.

12. The integrated device of claim 8, wherein the integrated device compares the results from the one or more oculomotor tests for a particular recognized individual against a baseline for the same individual previously recorded.

13. The integrated device of claim 1, wherein the mobile device is a mobile phone.

14. The integrated device of claim 1, wherein the integrated device measures a distance of the stimuli providing portion from one or more sensors using image recognition.

15. The integrated device of claim 1, wherein the integrated device measures a distance of the stimuli providing portion from one or more sensors using propagated electromagnetic waves.

16. The integrated device of claim 1, wherein the integrated device measures a distance of test subject from the sensors.

17. An apparatus comprising:
an add-on structure that communicates with a mobile device to provide supplemental features to the mobile device for conducting one or more oculomotor tests with the combination of the add-on structure and the mobile device, the add-on structure including a stimuli providing portion; and
an application loaded upon the mobile device to coordinate the interaction of the mobile device and add-on structure in the conducting of the oculomotor test.

18. The apparatus of claim 17,
wherein at least one of the add-on structure or the mobile device includes an infrared (IR) sensor and an at least one IR illuminator that provides information directly or indirectly to the application.

19. The apparatus of claim 18, wherein the IR sensor and the at least one IR illuminator (at a wavelength centered around 850 nm) are provided by the add-on structure.

20. The apparatus of claim 18, wherein the IR sensor on the add-on structure is in addition to a second IR sensor on the mobile device.

21. The apparatus of claim 17, wherein
the mobile device is a mobile phone with communication capabilities, and
the application is configured to utilize such communication capabilities communicate with remote computers for an account where information can be stored and recalled.

22. The apparatus of claim 17, wherein
the application instructs a user and automates at least a portion of the one or more oculomotor tests through feedback received through one or more sensors on the integrated device.

23. The apparatus of claim 17, wherein the stimuli-providing portion includes a plurality of light emitting diodes positioned on a screen, facilitating a Horizontal Gaze Nystagmus (HGN) procedure.

24. The apparatus of claim 23, wherein the screen is foldable, facilitating a Near Point Convergence (NPC) procedure.

25. The apparatus of claim 23, wherein the stimuli-providing portion includes an optimized pupillary light reflex (PLR)-related light flash.

26. The apparatus of claim 17, wherein the application utilizes one or more sensor from the combination of the add-on structure and the mobile device to recognize an individual.

27. The apparatus of claim 26, wherein the application compares the results from the one or more oculomotor tests for a particular recognized individual against a baseline for the same individual previously recorded.

28. The apparatus of claim 26, wherein one or both of the application and the add-on structure enables measurement of a distance of the stimuli providing portion from one or more sensors using image recognition.

29. The apparatus of claim 17, wherein one or both of the application and the add-on structure enables measurement of a distance of the stimuli providing portion from one or more sensors using propagated electromagnetic waves.

30. The apparatus of claim 17, wherein one or both of the application and the add-on structure enables measurement of a distance of test subject from one or more sensors.
